(19) United States
(12) Patent Application Publication
(10) Pub. No.: US 2026/0007549 A1
(43) Pub. Date: Jan. 8, 2026

(54) NURSING PAD WITH FILAMENT CORE AND PROCESS THEREOF

(71) Applicant: U-PLAY CORPORATION, Wuhu (CN)

(72) Inventors: Sheng Cheng, Wuhu (CN); Jing Jin, Wuhu (CN); Chao Lu, Wuhu (CN)

(21) Appl. No.: 18/813,585

(22) Filed: Aug. 23, 2024

(30) Foreign Application Priority Data
Jul. 2, 2024 (CN) 2024108785935

(51) Int. Cl. A61F 13/537; A61F 13/15; A61F 13/51; A61F 13/53

(52) U.S. Cl. CPC A61F 13/53704; A61F 2013/15146; A61F 2013/51047; A61F 2013/530029; A61F 2013/53791

(57) ABSTRACT

Nursing pad with a filament core and a process thereof, and relates to the technical field of nursing pads, comprising a layer of PE waterproof film and a layer of non-woven fabric, a cotton core is filled in the gap between the PE waterproof film and the non-woven fabric, filaments are blended inside the cotton core. The main highlight of the nursing pad in this invention is that the filaments are blended into the cotton core; filaments are a type of textile material, and during the filament spinning process, elastic fibers are added to make the filaments both flexible and elastic; in addition, during production, as the filaments are transported on the conveyor belt, the cotton core does not easily loosen, which enhances the equipment's operating speed and increases production output.

### 1. TECHNICAL FIELD

The invention relates to the technical field of nursing pads, in particular to a nursing pad with a filament core and a process thereof.

### 2. BACKGROUND ART

A nursing pad is a disposable sanitary product made from materials such as PE film, non-woven fabric, fluff pulp, and polymers. It includes diapers, reusable underpads, etc., and is primarily used for hospital surgeries, pet care, gynecological examinations, postpartum care, infant care, incontinence in paralyzed patients, and menstrual care for women.

For example, a diaper with deodorizing and antibacterial functions and a production process thereof, with a publication number CN112263392B and an authorization publication date of Apr. 1, 2022, features excellent absorption performance, quickly absorbing moisture within the diaper and maintaining its shape; additionally, it has a large liquid storage capacity, effectively preventing the liquid on the diaper from contacting the human body; it has nano antibacterial layer, providing antibacterial and sterilizing effects. However, there are still certain problems in the actual production and use of nursing pads, as detailed below.

The nursing pads currently circulating in the market are all designed with five layers, from bottom to top: 1. PE film (Polyethylene film, which is waterproof); 2. bottom tissue paper; 3. cotton core made by blending the wood pulp crushed by a crusher with water-absorbent polymers; 4. top tissue paper; 5. non-woven fabric. Among them, the main liquid-absorbing part is the cotton core. The tissue paper is placed on both upper and lower layers of the cotton core; the primary function of the tissue paper is to restrict the movement of the cotton core, preventing the cotton core from loosening. However, in reality, the design of the tissue paper on the top and bottom layers is primarily to meet production requirements; during the production of urine pads, the cotton core is made by blending wood pulp crushed by a crusher with water-absorbent polymers; this process and the transportation steps involved can cause a significant amount of lint to fly around and the cotton core to shift out of position; in order to improve production efficiency and reduce lint splashing, the tissue paper is added to the top and bottom layers of the cotton core to prevent the cotton core from shifting and loosening during production; however, the tissue paper layers only serve to stabilize the cotton core when the urine pad is unused; once the urine pad is in use, the tissue paper will break apart under the erosion of urine and the pressure and rubbing from the human body; therefore, during use, the tissue paper not only fails to function as a limiter, but also forms clumps within the cotton core after being eroded by urine and broken down; based on common life experience, tissue paper becomes hard after absorbing water and forming clumps, causing a strong foreign body sensation for users; furthermore, the broken tissue paper exacerbates the problem of the cotton core shifting and loosening, which in turn aggravates the uneven distribution of urine.

To address the above issues, there is an urgent need for innovative design based on the existing nursing pad and its manufacturing.

### 3. SUMMARY OF THE INVENTION

The technical scheme of the invention aims at the technical problem that the existing technical scheme is too single, and provides a scheme that is significantly different from the existing technology. Specifically, the object of the invention is to provide a nursing pad with a filament core and a process thereof to solve the problems of the above background technology. The problems of the above background technology are as follows: the design of the tissue paper on the top and bottom layers is primarily to meet production requirements; during the production of urine pads, the cotton core is made by blending wood pulp crushed by a crusher with water-absorbent polymers; this process and the transportation steps involved can cause a significant amount of lint to fly around and the cotton core to shift out of position; in order to improve production efficiency and reduce lint splashing, the tissue paper is added to the top and bottom layers of the cotton core to prevent the cotton core from shifting and loosening during production; however, the tissue paper layers only serve to stabilize the cotton core when the urine pad is unused; once the urine pad is in use, the tissue paper will break apart under the erosion of urine and the pressure and rubbing from the human body; therefore, during use, the tissue paper not only fails to function as a limiter, but also forms clumps within the cotton core after being eroded by urine and broken down; based on common life experience, tissue paper becomes hard after absorbing water and forming clumps, causing a strong foreign body sensation for users; furthermore, the broken tissue paper exacerbates the problem of the cotton core shifting and loosening, which in turn aggravates the uneven distribution of urine.

In order to achieve the above object, the invention provides the following technical scheme: a nursing pad with a filament core, comprising a layer of PE waterproof film and a layer of non-woven fabric, wherein a cotton core is filled in the gap between the PE waterproof film and the non-woven fabric, filaments are blended inside the cotton core.

Preferably, the PE waterproof film is located at a bottom of the nursing pad to prevent urine from leaking out.

Preferably, the non-woven fabric is positioned at a top of the nursing pad to allow urine to pass through and enhance surface flexibility.

Preferably, the filaments are made of flexible and antibacterial materials, and elastic fibers are blended within the filaments, giving them both flexibility and a certain degree of elasticity.

Preferably, the filaments are processed and formed by the interlocking and bridging of several sets of spiral filamentous structures, the cotton core is evenly dispersed and distributed within the gaps of the spiral filamentous structures of the filaments.

A process for producing the nursing pad with the filament core comprises the following steps:
S1: material feeding station: a release end of the PE waterproof film is unrolled by a unwinding mechanism and laid on a conveyor belt, then the PE waterproof film is transported at a uniform speed;
S2: cotton core processing station: the cotton core is formed by mixing wood pulp crushed by a crusher and water-absorbent polymers, then, the cotton core is laid on the PE waterproof film transported by the conveyor belt at a fixed time and interval according to the designated area;
S3: filament adding station: the cotton core continues to be transported along with the PE waterproof film to the filament adding station, at this time, the conveyor belt stops, and a pressing mechanism above compresses and positions the cotton core, ensuring the cotton core covers the PE waterproof film with a thickness of approximately 0.5-0.6 cm; subsequently, the filaments are drawn through spiral channels, so that the filaments are spirally inserted into an interior of the cotton core, the drawing process proceeds sequentially from the left station to the right station, with the overlapping range between adjacent spiral filaments designed to be 1-1.5 mm; once the filaments reach sufficient length, they are automatically cut off, the filament adding step is completed;
S4: covering station: after the filament adding step is completed, the cotton core and its carrier, the PE waterproof film, enter the covering station together and are gradually covered by the unwinding non-woven fabric; subsequently, edge sealing is performed;
S5: cutting station: after the covering and the edge sealing, the nursing pad, which is a combination of the PE waterproof film, the non-woven fabric, the cotton core, and the filaments, falls into the cutting station for fixed-length cutting; finally, the nursing pad undergoes packaging.

Compared with the prior art, the invention has the following advantageous effects:
1. the main highlight of the nursing pad in this invention is that the filaments are blended into the cotton core; filaments are a type of textile material, and during the filament spinning process, elastic fibers are added to make the filaments both flexible and elastic; in addition, during production, as the filaments are transported on the conveyor belt, the cotton core does not easily loosen, which enhances the equipment's operating speed and increases production output; additionally, the filaments are blended into the cotton core during production process, which ensures the stability of the cotton core during the production process, but also ensures the stability of the cotton core when the nursing pad is used; moreover, it eliminates the need for adding upper and lower layers of tissue paper, thereby streamlining the processing steps;
2. in this invention, the filaments are processed and formed by the interlocking and bridging of several sets of spiral filamentous structures; the spiral filaments are interconnected and pulled together, so during the use of the nursing pad, the pressure exerted on a specific area is distributed by the spiral filaments to various locations; this prevents the cotton core in that area from being compacted and unable to absorb urine, ensuring that the urine is evenly distributed throughout the pad; additionally, the spiral filaments mixed into the cotton core exert a good pulling and repositioning effect on the surrounding cotton core, allowing any area of the cotton core to rebound and return to its original shape even after prolonged compression, thereby providing a better user experience.

### 4. BRIEF DESCRIPTION OF ACCOMPANY DRAWINGS

FIG. 1 is a schematic diagram of the front cross-sectional structure of the invention;

FIG. 2 is an enlarged schematic diagram of the structure of point A in FIG. 1 of the invention;

FIG. 3 is a schematic diagram of the top view structure of the invention;

FIG. 4 is a schematic diagram of the top cross-sectional structure of the invention;

FIG. 5 is a schematic diagram of the three-dimensional structure of the filaments of the invention.

In the figures: 1 PE waterproof film; 2 non-woven fabric; 3 cotton core; 4 filament.

### 5. SPECIFIC EMBODIMENT OF THE INVENTION

The technical schemes in the embodiments of the invention will be clearly and completely described in combination with the accompanying drawings in the embodiments of the invention. Obviously, the described embodiments are only some of the embodiments of the invention, but not all of the embodiments. Based on the embodiments in this invention, all other embodiments obtained by those of ordinary skill in the art without making creative efforts shall fall within the protection scope of this invention.

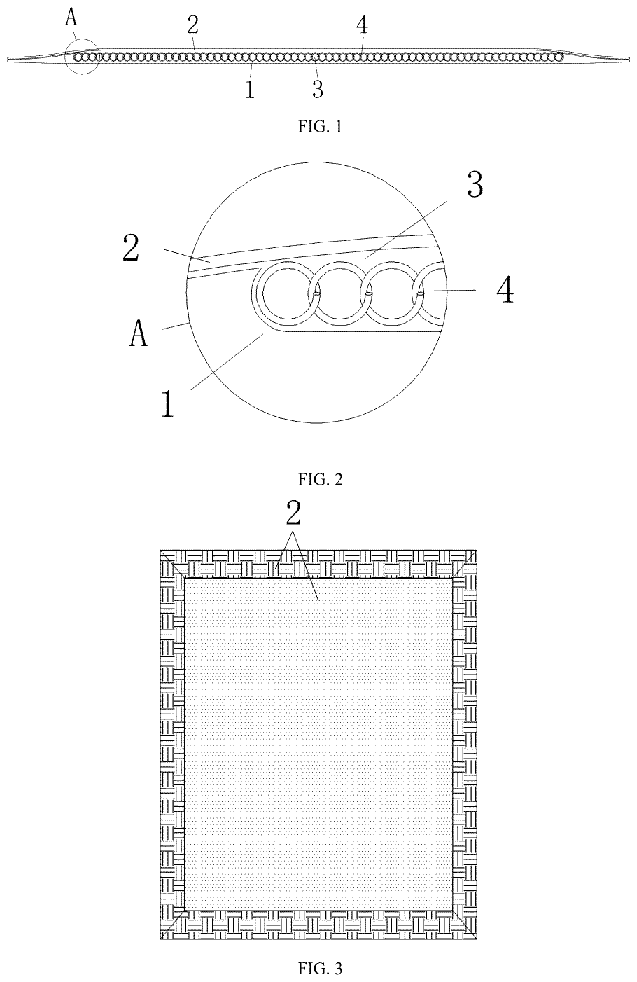

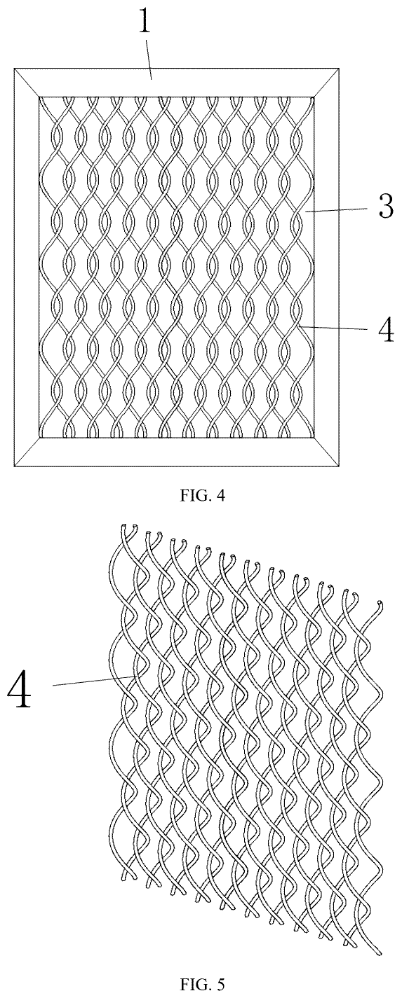

Please refer to FIG. 1-5, the invention provides the following technical scheme: a nursing pad with a filament core comprises a layer of PE waterproof film 1 and a layer of non-woven fabric 2, wherein a cotton core 3 is filled in the gap between the PE waterproof film 1 and the non-woven fabric 2, filaments 4 are blended inside the cotton core 3.

The PE waterproof film 1 is located at a bottom of the nursing pad to prevent urine from leaking out.

The non-woven fabric 2 is positioned at a top of the nursing pad to allow urine to pass through and enhance surface flexibility.

The filaments 4 are made of flexible and antibacterial materials, and elastic fibers are blended within the filaments 4, giving them both flexibility and a certain degree of elasticity.

The filaments 4 are processed and formed by the interlocking and bridging of several sets of spiral filamentous structures, the cotton core 3 is evenly dispersed and distributed within the gaps of the spiral filamentous structures of the filaments 4.

A process for producing the nursing pad with the filament core comprises the following steps:
S1: material feeding station: a release end of the PE waterproof film 1 is unrolled by a unwinding mechanism and laid on a conveyor belt, then the PE waterproof film 1 is transported at a uniform speed;
S2: cotton core processing station: the cotton core 3 is formed by mixing wood pulp crushed by a crusher and water-absorbent polymers, then, the cotton core 3 is laid on the PE waterproof film 1 transported by the conveyor belt at a fixed time and interval according to the designated area;
S3: filament adding station: the cotton core 3 continues to be transported along with the PE waterproof film 1 to the filament adding station, at this time, the conveyor belt stops, and a pressing mechanism above compresses and positions the cotton core 3, ensuring the cotton core 3 covers the PE waterproof film 1 with a thickness of approximately 0.5-0.6 cm; subsequently, the filaments 4 are drawn through spiral channels, so that the filaments 4 are spirally inserted into an interior of the cotton core 3, the drawing process proceeds sequentially from the left station to the right station, with the overlapping range between adjacent spiral filaments designed to be 1-1.5 mm; once the filaments reach sufficient length, they are automatically cut off, the filament adding step is completed;
S4: covering station: after the filament adding step is completed, the cotton core 3 and its carrier, the PE waterproof film 1, enter the covering station together and are gradually covered by the unwinding non-woven fabric 2; subsequently, edge sealing is performed;
S5: cutting station: after the covering and the edge sealing, the nursing pad, which is a combination of the PE waterproof film 1, the non-woven fabric 2, the cotton core 3, and the filaments 4, falls into the cutting station for fixed-length cutting; finally, the nursing pad undergoes packaging.

The main highlight of the nursing pad in this invention is that the filaments are blended into the cotton core; filaments are a type of textile material, and during the filament spinning process, elastic fibers are added to make the filaments both flexible and elastic; in addition, during production, as the filaments are transported on the conveyor belt, the cotton core does not easily loosen, which enhances the equipment's operating speed and increases production output; additionally, the filaments are blended into the cotton core during production process, which ensures the stability of the cotton core during the production process, but also ensures the stability of the cotton core when the nursing pad is used; moreover, it eliminates the need for adding upper and lower layers of tissue paper, thereby streamlining the processing steps; in this invention, the filaments are processed and formed by the interlocking and bridging of several sets of spiral filamentous structures; the spiral filaments are interconnected and pulled together, so during the use of the nursing pad, the pressure exerted on a specific area is distributed by the spiral filaments to various locations; this prevents the cotton core in that area from being compacted and unable to absorb urine, ensuring that the urine is evenly distributed throughout the pad; additionally, the spiral filaments mixed into the cotton core exert a good pulling and repositioning effect on the surrounding cotton core, allowing any area of the cotton core to rebound and return to its original shape even after prolonged compression, thereby providing a better user experience.

Although the invention has been described in detail with reference to the aforementioned embodiments, it is still possible for those skilled in the art to modify the technical schemes described in the aforementioned embodiments, or to replace some of the technical features therein with equivalents. Any modification, equivalent replacement, improvement, etc. made within the spirit and principle of the invention shall be included in the protection scope of the invention.

1. A nursing pad with a filament core, comprising a layer of PE waterproof film and a layer of non-woven fabric, wherein a cotton core is filled in the gap between the PE waterproof film and the non-woven fabric, filaments are blended inside the cotton core.

2. The nursing pad with the filament core of claim 1, wherein the PE waterproof film is located at a bottom of the nursing pad to prevent urine from leaking out.

3. The nursing pad with the filament core of claim 1, wherein the non-woven fabric is positioned at a top of the nursing pad to allow urine to pass through and enhance surface flexibility.

4. The nursing pad with the filament core of claim 1, wherein the filaments are made of flexible and antibacterial materials, and elastic fibers are blended within the filaments, giving them both flexibility and a certain degree of elasticity.

5. The nursing pad with the filament core of claim 1, wherein the filaments are processed and formed by the interlocking and bridging of several sets of spiral filamentous structures, the cotton core is evenly dispersed and distributed within the gaps of the spiral filamentous structures of the filaments.

6. A process for producing the nursing pad with the filament core of claim 1, comprising the following steps:
S1: material feeding station: a release end of the PE waterproof film is unrolled by a unwinding mechanism and laid on a conveyor belt, then the PE waterproof film is transported at a uniform speed;
S2: cotton core processing station: the cotton core is formed by mixing wood pulp crushed by a crusher and water-absorbent polymers, then, the cotton core is laid on the PE waterproof film transported by the conveyor belt at a fixed time and interval according to the designated area;
S3: filament adding station: the cotton core continues to be transported along with the PE waterproof film to the filament adding station, at this time, the conveyor belt stops, and a pressing mechanism above compresses and positions the cotton core, ensuring the cotton core covers the PE waterproof film with a thickness of approximately 0.5-0.6 cm; subsequently, the filaments are drawn through spiral channels, so that the filaments are spirally inserted into an interior of the cotton core, the drawing process proceeds sequentially from the left station to the right station, with the overlapping range between adjacent spiral filaments designed to be 1-1.5 mm; once the filaments reach sufficient length, they are automatically cut off, the filament adding step is completed;
S4: covering station: after the filament adding step is completed, the cotton core and its carrier, the PE waterproof film, enter the covering station together and are gradually covered by the unwinding non-woven fabric; subsequently, edge sealing is performed;
S5: cutting station: after the covering and the edge sealing, the nursing pad, which is a combination of the PE waterproof film, the non-woven fabric, the cotton core, and the filaments, falls into the cutting station for fixed-length cutting; finally, the nursing pad undergoes packaging.
